(19) United States
(12) Patent Application Publication
(10) Pub. No.: US 2006/0009787 A1
(43) Pub. Date: Jan. 12, 2006

(54) Simplified stereotaxtic brain surgery device, and band used for determining position at which such device is mounted on patient head

(76) Inventor: Hiroyuki Tai, Toyonaka-shi (JP)

Correspondence Address: KIRK HAHN, 14431 HOLT AVE, SANTA ANA, CA, 92705, US

(21) Appl. No.: 10/526,805

(22) PCT Filed: Aug. 26, 2003
(86) PCT No.: PCT/JP03/10804

§ 371 Date: Mar. 5, 2005

(30) Foreign Application Priority Data
Sep. 6, 2002 (JP) 2002-262054

(51) Int. Cl. A61B 19/00

(52) U.S. Cl. 606/130

(57) ABSTRACT

A simplified stereotactic brain surgery device (1) comprises a frame (2) with puncture guides for guiding the tip of a puncturing needle to a predetermined position within the brain, and right and left fixing frames (3, 4) respectively having fixing needles (31, 41) for fixing the device (1) on the patient head, the fixing frames (3, 4) being displaceable in a longitudinal direction of the frame (2), and the frame (2) being provided with a plurality of guide grooves (21) for guiding the tip of a puncturing needle toward a point on a line connecting the right and left fixing needles (31, 41).

### TECHNICAL FIELD

The present invention relates to simplified stereotactic brain surgery devices used for surgery on cerebral diseases such as hypertensive cerebral hemorrhages, brain tumors, brain abscesses, hydrocephalus, or cerebral cysts, and to bands used for determining the position at which such a device is mounted on a patient's head.

### BACKGROUND ART

Conventionally, so-called Komai-type CT stereotactic brain surgery devices (referred to as “Komai-type devices” in the following) are known as stereotactic brain surgery devices (see Document 1 below, for example). The Komai-type devices had hygienic problem, because it is necessary to mount the device from the stage of taking the CT images prior to surgery.

Document 1: Hiroshi Abe, Ryuichi Tanaka, Kimiyoshi Hayakawa, Haruhiko Kikuchi, Takashi Tsubokawa, Satoru Matsumoto (ed.) “NOUSHINKEIGEKASHIKKAN-NO SHUJUTSU TO TEKIOU II” (“Surgery of Neurosurgical Diseases and its Application II”), Asakura Shoten, Oct. 15, 1990, First Ed., pp. 237-245.

Furthermore, the following Document 2 proposes a simplified stereotactic brain surgery device with a simpler configuration. This device requires for example an arc-shaped frame for the puncturing with a puncturing needle from the desired direction, which made its configuration complex. Furthermore, there were also disadvantages with regard to safety.

Document 2: JP H11-137568A (Published on May 25, 1999, in particular FIG. 1)

### DISCLOSURE OF THE INVENTION

It is an object of the present invention to provide a simplified stereotactic brain surgery device that can be manufactured easily and inexpensively, and that is hygienic with superior handling properties and safety, and moreover to provide a band that is used to determine the position where this device is mounted to a patient's head.

In order to achieve these objects, a simplified stereotactic brain surgery device according to the present invention comprises a guide frame for guiding a puncturing needle's tip to a predetermined location within a brain; and left and right fixing frames, each having a fixing member for fixing the device to the patient's head, the fixing frames being displaceable in a longitudinal direction of the guide frame; wherein the guide frame is provided with a plurality of guide depressions that guide the puncturing needle's tip to a point on the line connecting the left and right fixing members.

With this configuration, the guide depression that is used to guide the puncturing needle to a predetermined location within the brain (location of the lesion) can be selected from the plurality of guide depressions, so that the puncturing needle can be inserted from the optimum direction into the brain, and after first inserting a puncturing needle with a smaller diameter along the selected guide depression, a puncturing needle with a larger diameter can be inserted along the same guide depression, so that brain surgery with a higher degree of safety becomes possible. Furthermore, it is also possible to simultaneously insert a plurality of puncturing needles from different guide depressions.

It should be noted that here, “puncturing needle” refers to a structure having an outer cylinder and an inner cylinder, and with which drainage or evacuation can be performed from the tip, in an emptied state that is achieved by pulling out the inner cylinder. “Puncturing needle” includes a probe, for example.

It is preferable that the simplified stereotactic brain surgery device according to the present invention further comprises any of the following configurations.

(1) A configuration wherein the guide frame is provided with a scale. Thus, it is easy to position the left and right fixing frames, and to specify the position of the selected guiding depression, for example.

(2) A configuration wherein the guide frame comprises a displacement prevention member for preventing displacement of the puncturing needle when the puncturing needle is guided by one of the guide depressions. Thus, when a puncturing needle is inserted into the brain, a displacement of the puncturing needle can be reliably prevented, so that a device with high safety can be realized.

(3) The configuration of (2), further comprising a pressing member that presses the displacement prevention member against a main member of the guide frame. Thus, a displacement of the puncturing needle can be more reliably prevented.

(4) A configuration wherein an auxiliary fixing member for aiding fixation of the device on the patient's head is provided on at least one of the left and right fixing frames. Thus, the device can be reliably fixed to the patient's head.

(5) The configuration of (2), wherein the auxiliary fixing member comprises an auxiliary fixing portion for aiding fixation of the device on the patient's head, a linking member linking the auxiliary fixing portion and the fixing frame at an adjustable angle with the fixing member at the center, and a distance adjustment member for adjusting a distance between the auxiliary fixing portion and that fixing member. Thus, the position where the device is fixed to the patient's head with the auxiliary fixing member can be freely selected within the adjustment range. That is to say, the range over which fixing with the auxiliary fixing member is possible is broad, and a safer fixing position can be selected.

In accordance with the present invention, a band that is used prior to brain surgery using the simplified stereotactic brain surgery device according to the present invention, to determine a position where the device is mounted to the patient's head and to mark this position, has a plurality of marker members that are arranged at opposing positions when the band is wrapped around the patient's head. It is preferable that the marker members comprise aluminum or an alloy thereof, stainless steel, brass, copper, nichromium, processed animal bone, or tooth. If the marker members are made from any of these materials, then the positions of the marker members when taking a CT or the like can be clearly visible in the form of dots, and it is possible to easily work for determining the device mounting position on the patient's head.

It is preferable that the main member of the band is mesh-shaped. Thus, it is possible to write marks from above the band onto the patient's head around which the band is wrapped.

These and other objects, features and advantages of the present invention will become more apparent from the following description. Also, beneficial results of the present invention should be obvious from the following description when taken in conjunction with the accompanying drawings.

### BRIEF DESCRIPTION OF THE DRAWINGS

FIG. 1 is an exploded perspective view showing the overall configuration of a simplified stereotactic brain surgery device according to an embodiment of the present invention.

FIG. 2 shows a front view and a side view of this simplified stereotactic brain surgery device in an assembled state.

FIG. 3 is an enlarged view of this simplified stereotactic brain surgery device showing how a puncturing needle is inserted between a main member of a frame with puncture guides and a displacement prevention plate.

FIGS. 4(a) to 4(d) show front views and rear views of the two bands constituting a marking band according to an embodiment of the present invention.

FIG. 5 is a diagram showing the configuration of a marking band.

FIGS. 6(a) to 6(c) illustrate a step of applying markings to the patient's head prior to the operation, using this marking band.

FIGS. 7(a) and 7(b) illustrate a step of applying markings to the patient's head prior to the operation, using this marking band.

FIGS. 8(a) and 8(b) are diagrammatic views of CT images and illustrate a step of determining the device mounting position.

FIGS. 9(a) and 9(b) illustrate a step of applying markings to the patient's head prior to the operation, using this marking band.

FIGS. 10(a) and 10(b) are diagrammatic views of CT images and illustrate a step of determining the distance between the left and right device mounting positions and the center of the lesion.

FIGS. 11(a) and (b) are diagrams showing the operating field.

FIG. 12 is a diagram illustrating the step of setting the position of the left fixing frame of the simplified stereotactic brain surgery device

FIG. 13 is a diagram illustrating a step of mounting the simplified stereotactic brain surgery device to the patient's head.

FIG. 14 is a diagram illustrating a step of selecting the optimum guide groove before inserting the puncturing needle from the perforation location into the brain.

FIG. 15 is a diagram showing the state in which the simplified stereotactic brain surgery device is fixed, with the auxiliary fixing support, in the position that is optimal for puncturing

FIG. 16 is a diagram showing a stopper and how this stopper is attached to a puncturing needle.

FIG. 17 is an exploded perspective view showing the overall configuration of a simplified stereotactic brain surgery device in accordance with another embodiment of the present invention.

### BEST MODE FOR CARRYING OUT THE INVENTION

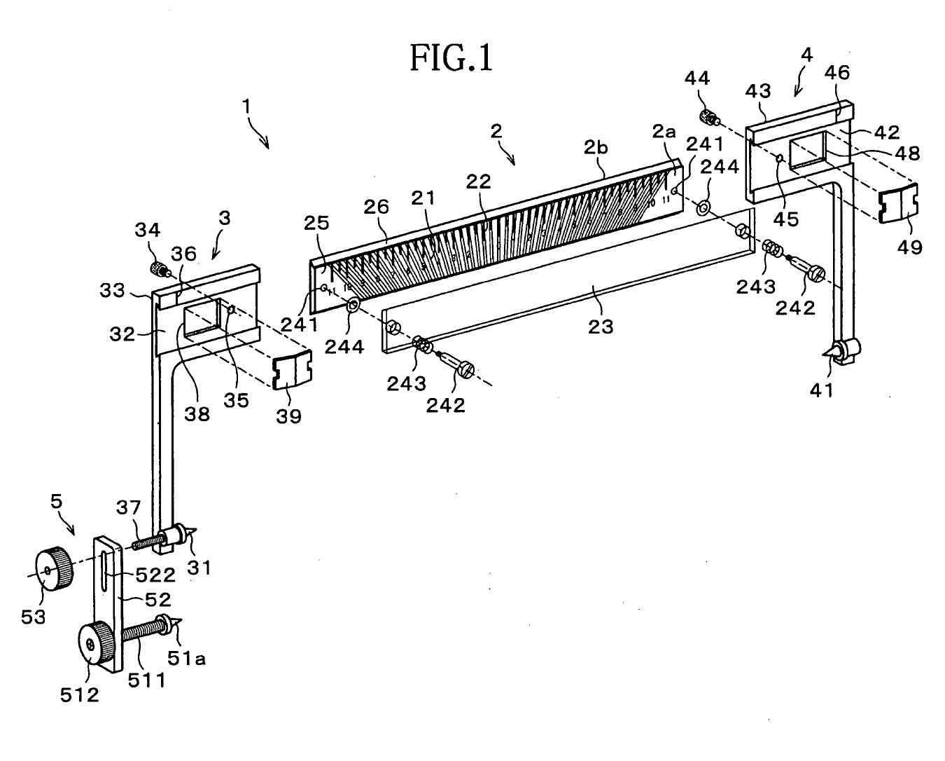

The following is an explanation of an embodiment of the present invention, with reference to FIGS. 1 to 17.

(1) Device Configuration of the Present Embodiment

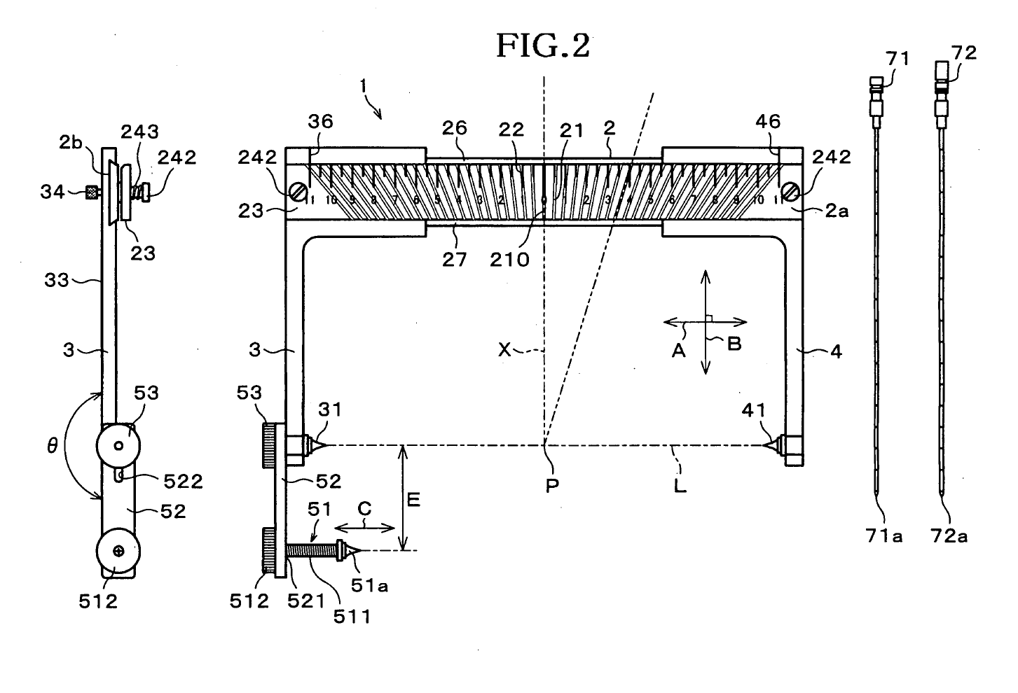

FIG. 1 is an exploded perspective view showing the overall configuration of a simplified stereotactic brain surgery device (also referred to as “the device” in the following) according to the present embodiment. FIG. 2 shows a front view and a side view of the device in an assembled state (also shown are two puncturing needles 71 and 72 with different diameters). The device 1 includes a frame 2 with puncture guides (guide frame), a right fixing frame 3, a left fixing frame 4, and an auxiliary fixing support (auxiliary fixing member) 5.

A plurality of guide grooves 21 (guide depressions) for guiding tips 71a and 72a of the puncturing needles 71 and 72 to the location of the lesion within the brain (in the later-described usage method, the hematoma corresponds to this lesion location) and scales (scale marks) 22 that are used, for example, for positioning the left and right fixing frames 3 and 4 are engraved in the frame 2 with puncture guides. The scales 22 have a point of origin “0” in their center, and a set of marks with 5 mm intervals to the left and right until 11 cm on the left and right.

The guide grooves 21 are provided in the same number as the scales 22, and of these, the guide groove 210 that is located at the point of origin “0” is engraved in a direction B perpendicular to an arrow direction A, which is a longitudinal direction of the frame 2 with puncture guides (in other words, the direction B is perpendicular to a line L connecting later-described left and right fixing needles 31 and 41). The directions of the guide grooves 21 other than this guide groove 210 are all engraved in a direction pointing to an intersection point P at which a line X extending from the groove direction of the guide groove 210 intersects with the line L connecting the left and right fixing needles 31 and 41. That is to say, all of the guide grooves 21 on the frame 2 with puncture guides are formed in a radiating pattern with the intersection point P at the center, such that lines extending from groove directions converge at the intersection point P.

On a surface 2a onto which the guide grooves 21 and the scales 22 are engraved, the frame 2 with puncture guides has a displacement prevention plate (displacement prevention member) 23 for preventing displacement when inserting the puncturing needles 71, 72. The displacement prevention plate 23 is formed transparently, so that the guide grooves 21 and the scales 22 can be viewed through this displacement prevention plate 23. The displacement prevention plate 23 is attached to the main member 25 of the frame 2 with puncture guides by screws 242 that are screwed into left and right screw holes 241, coil springs 243 (pressure members), and washers 244. Thus, there is a gap of the thickness of the washer 244 between the displacement prevention plate 23 and the main member 25, and the displacement prevention plate 23 is pressed against the main member 25 of the frame 2 with puncture guides by the spring force of the coil spring 243. Consequently, even when either of the two puncturing needles 71 and 72 of different diameters is inserted between the main member 25 and the displacement prevention plate 23, the puncturing needle 71 or 72 is gripped tight, and its displacement can be prevented securely.

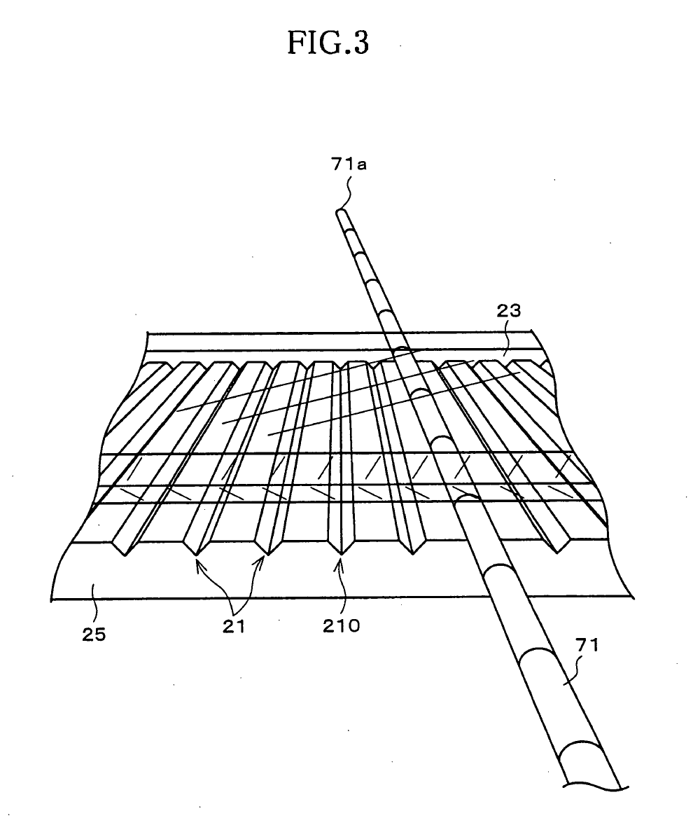

FIG. 3 is an enlarged view showing how the puncturing needle 71 is inserted between the main member 25 of the frame 2 with puncture guides and the displacement prevention plate 23. As shown in the figure, the puncturing needle 71 is sandwiched by the main member 25 and the displacement prevention plate 23, and is securely inserted along one of the guide grooves 21. Consequently, displacement of the puncturing needle 71 during insertion can be prevented, and the tip 71a of the puncturing needle 71 can reach the lesion location in the brain securely. It should be noted that in FIG. 3, the scales 22 have been omitted to keep the explanations simple.

Referring again to FIG. 1 and FIG. 2, one end 26 and the other end 27 of the frame 2 with puncture guides are tapered (that is, widening from a front surface 2a to a rear surface 2b) and the main member 25 of the frame 2 with puncture guides is provided with a substantially trapezoidal cross section. Depressions 32 and 42, into which this trapezoid shape can be fitted, are formed in the left and right fixing frames 3 and 4. Consequently, the fixing frames 3 and 4 are configured such that they are freely shiftable in the longitudinal direction of the frame 2 with puncture guides (that is, in the direction of arrow A), by fitting the main member 25 of the frame 2 with puncture guides into the depressions 32 and 42 of the fixing frames 3 and 4 and sliding.

When the left and right fixing frames 3 and 4 are to be fixed in a given position on the frame 2 with puncture guides, they can be easily fixed by tightening the fixing screws 34 and 44 from rear sides 33 and 43 of the fixing frames 3 and 4 at that position. That is to say, the fixing frames 3 and 4 can be easily fixed at that position by passing the fixing screws 34 and 44 through screw holes 35 and 45 of the fixing frames 3 and 4 and tightening the fixing screws 34 and 44 against the rear side 2b of the frame 2 with puncture guides.

As shown in FIG. 1, the depressions 32 and 42 of the left and right fixing frames 3 and 4 are further provided with substantially rectangular depressions 38 and 48 (which are depressions and do not reach through to the rear sides 33 and 43), and slide-stopping members 39 and 49 are fitted into these rectangular depressions 38 and 48. The slide-stopping members 39 and 49 have a substantially planar shape, but are slightly tilted from the left and right toward the center, and they are formed such that when they are fitted into the rectangular depressions 38 and 48, the center of the slide-stopping members 39 and 49 protrudes slightly from the surface of the depressions 32 and 42. Thus, once the fixing frames 3 and 4 have been fixed to the frame 2 with puncture guides, easy shifting of the fixing frames 3 and 4 is prevented, and even if the fixing screws 34 and 44 are loosened, easy positional displacement of the fixing frames 3 and 4 can be prevented.

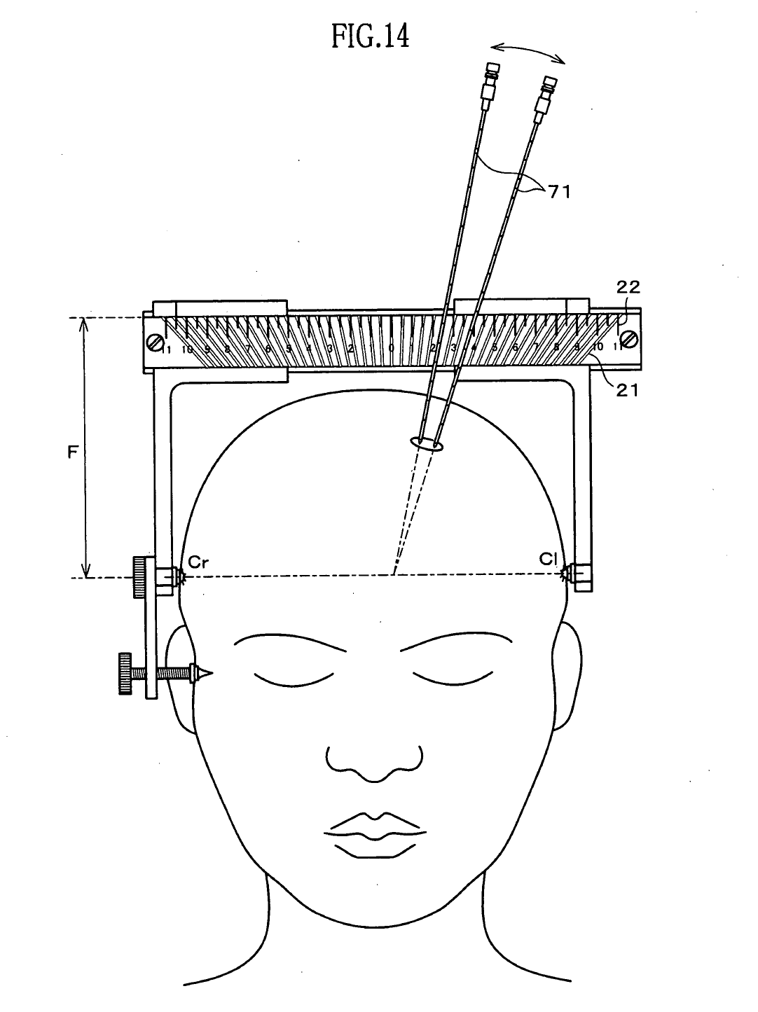

The left and right fixing frames 3 and 4 have fixing needles (fixing members) 31 and 41 at their ends. The fixing needles 31 and 41 are for mounting the device 1 to the patient's head, and are fixed to the patient's cranium at a later-described device mounting location Cr, Cl (see FIG. 14).

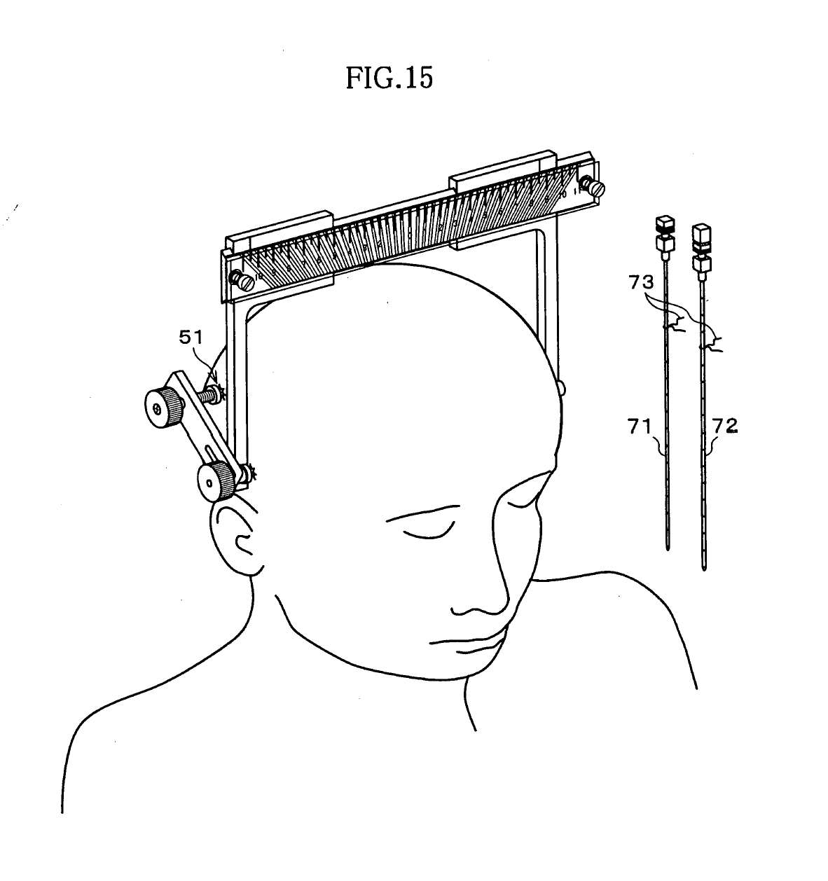

A male thread 37 is formed on the right fixing frame 3 on the side opposite to the fixing needle 31, and an auxiliary fixing support 5 can be attached to the right fixing frame 3 through this male thread 37. The auxiliary fixing support 5 is for assisting the mounting of the device 1 to the patient's cranium by three-point fixing (see FIG. 15). The auxiliary fixing support 5 includes an auxiliary fixing needle (auxiliary fixing portion) 51, a linking arm (linking member) 52, and a position fixing knob 53. The auxiliary fixing needle 51 has a threaded portion 511 and a knob portion 512. The threaded portion 511 is screwed into a screw hole 521 of the linking arm 52. Consequently, by rotating the knob portion 512, the tip 51a of the auxiliary fixing needle 51 can be shifted in the direction of arrow C (which is a direction parallel to the direction of arrow A).

One end of the linking arm 52 is provided with an oblong aperture portion 522 in longitudinal direction of the linking arm 52. The linking arm 52 is fixed to the right fixing frame 3 by turning and fastening the position fixing knob 53, which is screwed onto the male thread 37, while the male thread 37 is inserted with play into this aperture portion 522. Thus, when the position fixing knob 53 is loosened, the angle θ between the linking arm 52 and the right fixing frame 3 and the distance E between the fixing needle 31 and the auxiliary fixing needle 51 can be easily adjusted by fastening the position fixing knob 53 after setting the angle θ and the distance E as desired. That is to say, the position fixing knob 53 and the aperture portion 522 have the role of an adjusting portion for adjusting the angle θ and the distance E.

Scalp surface matching lines 36 and 46 are engraved into the left and right fixing frames 3 and 4. These scalp surface matching lines 36 and 46 are used to set the positions of the fixing frames 3 and 4 by adjusting them to scalp surface positions of the device mounting locations Cr and Cl. How these scalp surface matching lines 36 and 46 are used specifically is explained in more detail later.

The main member 25, the left and right fixing frames 3 and 4 and the auxiliary fixing support 5 of the device 1 are all made of stainless steel. The displacement prevention plate 23 is transparent, so that it is made of acrylic resin.

(2) Configuration of the Band of the Present Embodiment

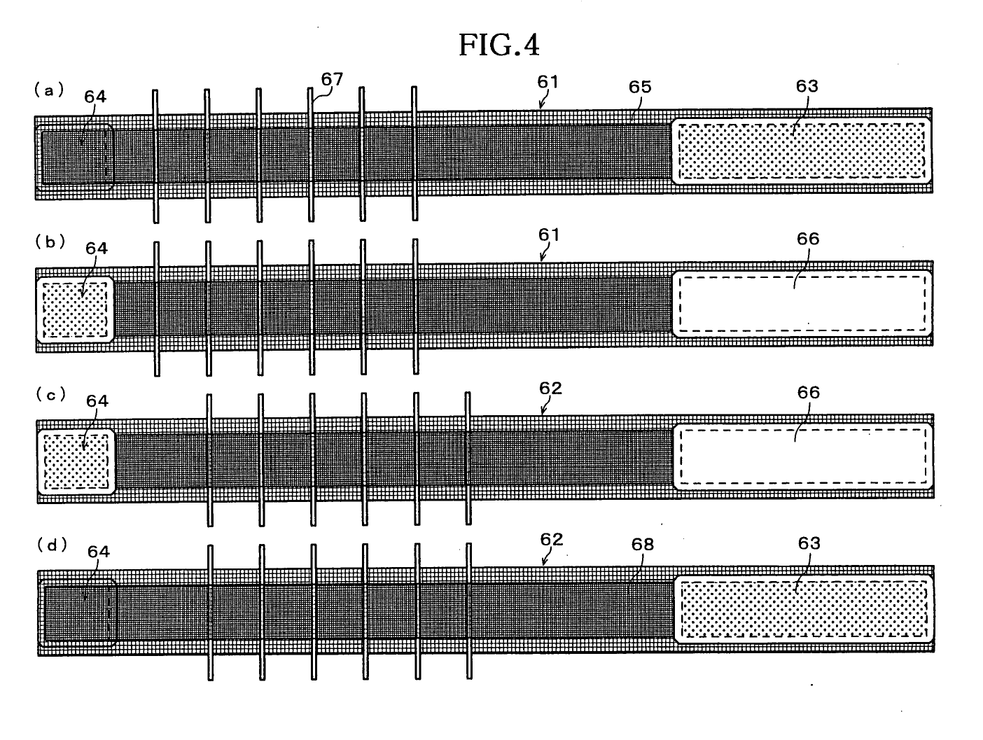

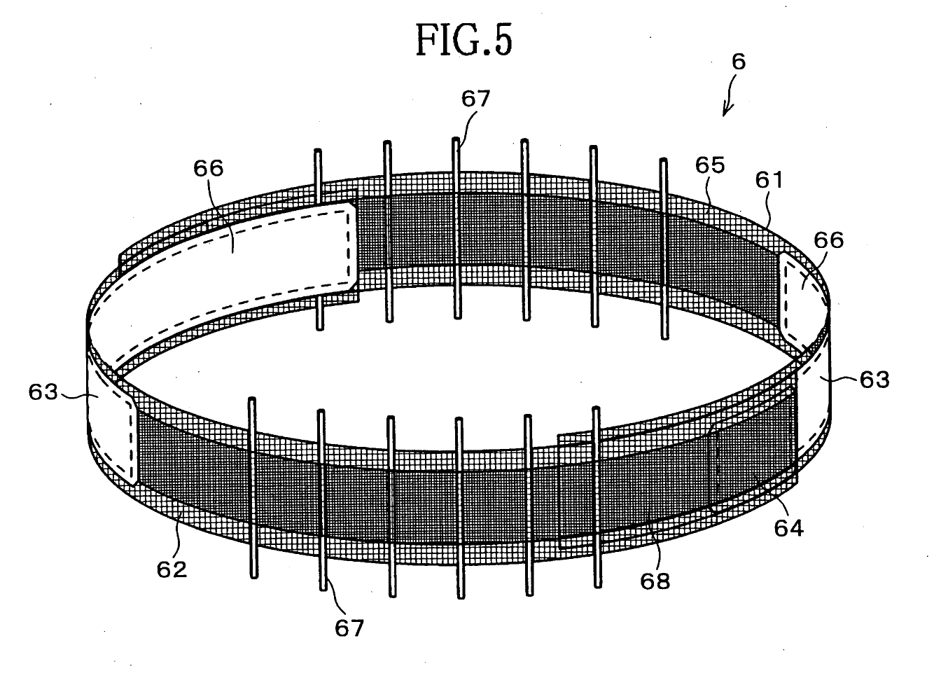

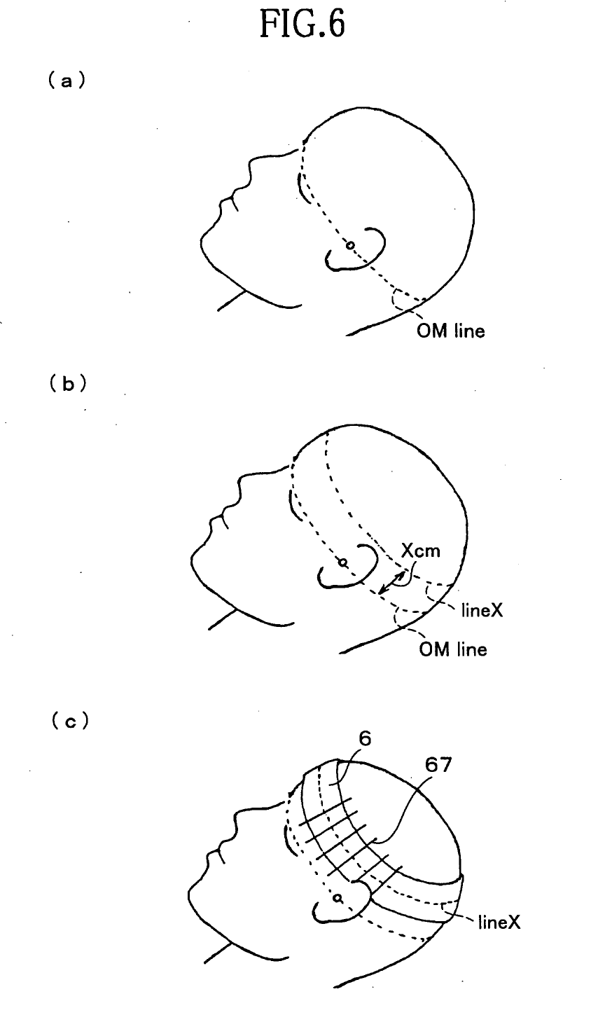

The following is an explanation of a marking band 6 for determining the mounting position of the device 1 on the patient's head, and for marking this position, prior to brain surgery using the device 1. As shown in FIG. 4 and FIG. 5, this marking band 6 is made of a combination of two bands 61 and 62, and is wrapped as a ring-shaped band around the patient's head by linking the respective ends of these two bands 61 and 62 together with cloth tapes 63 and 64 (see FIG. 6(c)). The cloth tapes 63 and 64 are Velcro™ type tapes, and can engage one another in a releasable manner. The cloth tapes 63 and 64 are sewn onto a main member 65 of the two bands 61 and 62. The cloth tape 63 is formed on an oblong fashion along the main member 65, such that the length of the ring formed by the marking band 6 can be easily adjusted to the size of the patient's head. Furthermore, a strengthening fabric 66 is sewn onto the reverse side of the main member 65 onto which the cloth tape 63 is sewn, in order to strengthen the marking band 6 and prevent it from slipping.

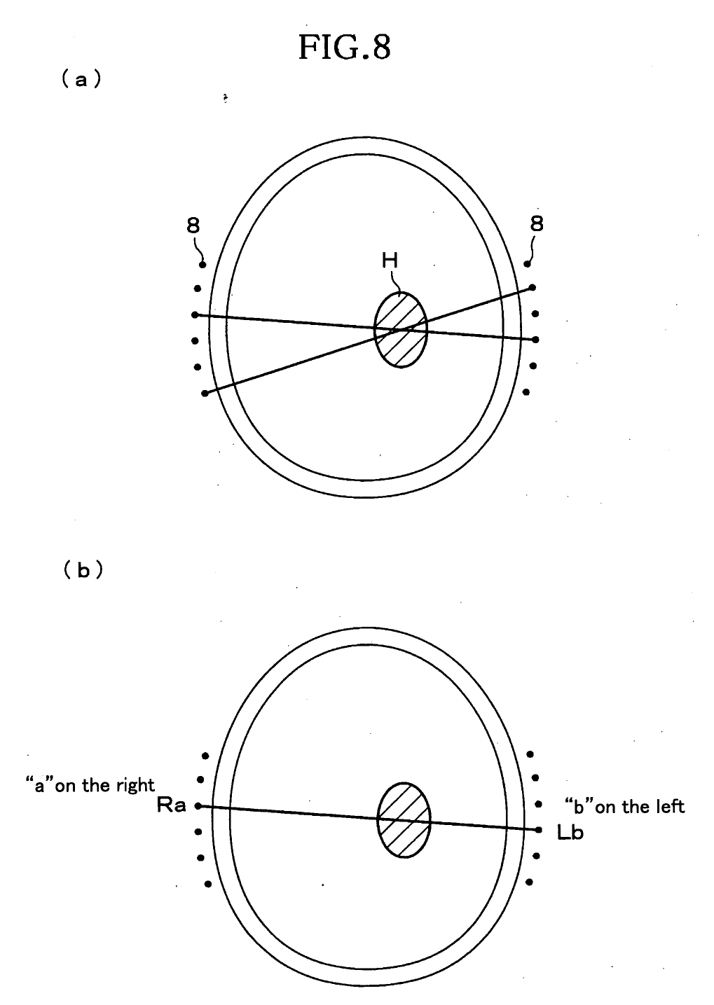

The two bands 61 and 62 are provided with six mark sticks (marker members) 67 at equal intervals. These mark sticks 67 are used in order to determine the mounting location of the device 1 on the patient's head using CT or the like. How these mark sticks 67 are used specifically is explained in more detail later. The marking band 6 is wrapped around the patient's head such that the six mark sticks 67 provided on the two bands 61 and 62 are located at positions in opposition to one another. The length of the mark sticks 67 is about 5 cm, and they are made of metal rods from an aluminum alloy having flexibility. By using such a material, the positions of the mark sticks 67 appear as clear dots on the photograph when taking a CT or the like (see FIGS. 8(a) and 8(b)).

The main members of two bands 61 and 62 are formed as a mesh, so that when wrapped around the patient's head, markings can be applied to the patient's head (marks can be written) from above the marking band 6. Moreover, a central portion 68 of the two bands 61 and 62 is made of a double-layered mesh material, in order to provide the marking band 6 with a given strength, and so that the mark sticks 67 can be easily inserted into the main members 65. In view of its strength, this mesh material is not made of a natural fiber, but of a synthetic fiber.

(3) Method for Using the Device and the Band of the Present Embodiment

The following is an explanation of an example of a method for using the device 1 and the marking band 6, divided into the marking stage prior to the operation and the operation stage of evacuation surgery of a cerebral hematoma.

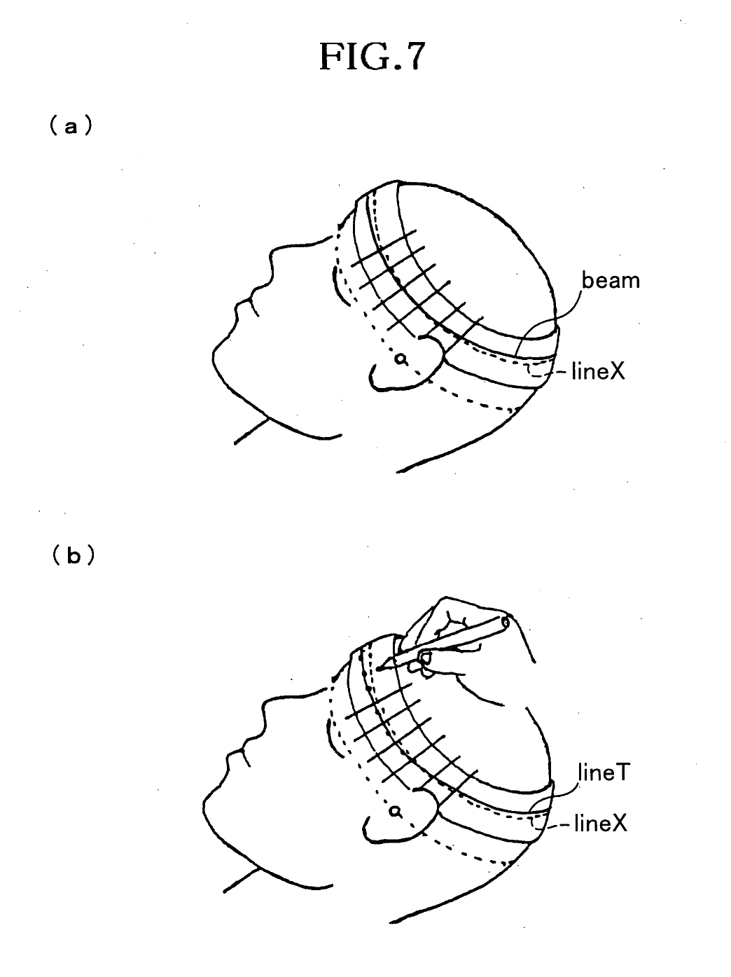

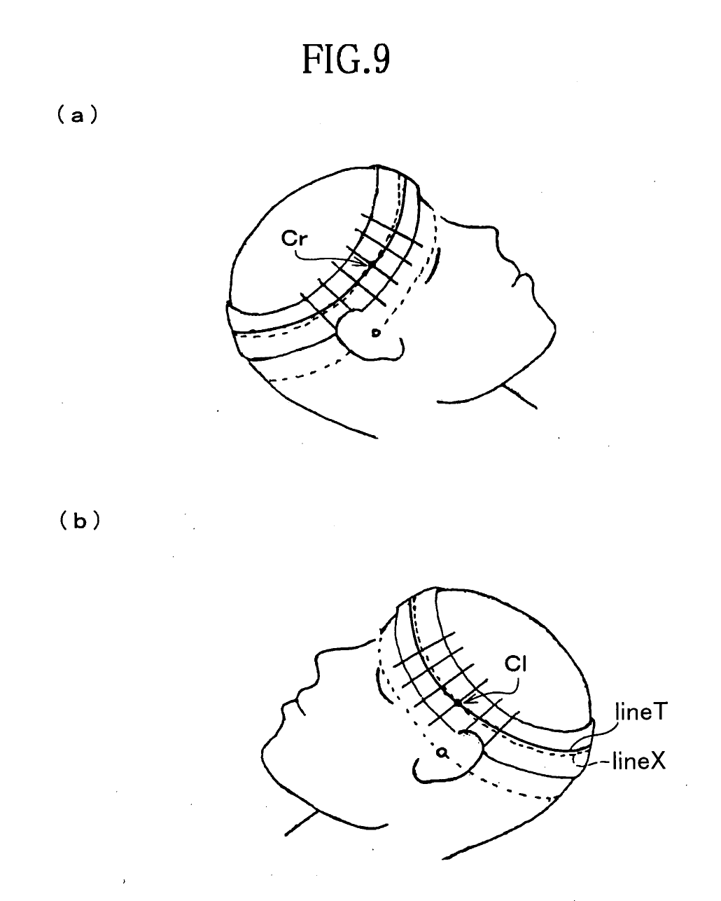

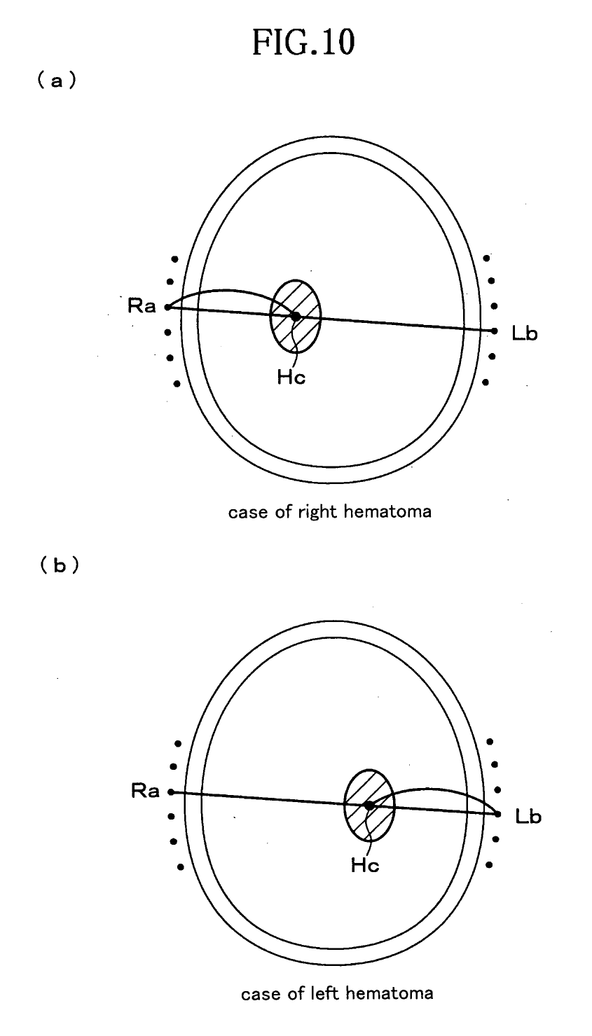

A. The Marking Stage Prior to the Operation

[Step A-1] Which slice has the maximum diameter of the cerebral hematoma is known from the cerebral CT that was previously taken for diagnosis (or taken for pre-surgery evaluation). It is read and confirmed how many centimeters this slice is above (or below) the OM line (the line extending from the eyeball to the external auditory canal). This value is taken as X cm (see FIG. 6(a) and (b)).

[Step A-2] The OM line is determined on the patient's head (which must be completely shaved) and marked. The line at X cm above (or below) and parallel to this OM line is also marked (referred to as “line X”) (see FIG. 6(a) and (b)). This line X does not necessarily have to be drawn with great accuracy on the head, because the mark sticks 67 of the marking band 6 cover a total vertical length of 5 cm.

[Step A-3] The marking band 6 is stored with the two bands 61 and 62 still separate, and the mark sticks 67 not inserted. Accordingly, first, the mark sticks 67 are inserted into the two bands 61 and 62, and then the marking band 6 is wrapped around the patient's head with the line X at the center, as shown in FIG. 6(c).

[Step A-4] The patient is moved to the CT room, and the patient's head is fixed inside the CT gantry. At this time, care is taken that the marking band 6 is not hidden by a band installed on the CT gantry, so that the patient's head can be irradiated with a beam in the following Step A-7.

[Step A-5] CT images are taken.

[Step A-6] After taking the CT, the slice in which the hematoma is shown largest (this is also referred to as the target slice or “T slice” below) is displayed on the CT monitor.

[Step A-7] A beam matching that of the T slice (for example the tenth slice) is irradiated onto the patient's head from the CT gantry by a radiological technician or the like, and the line on which the beam is incident (referred to as the line T) is marked on the head from above the marking band 6 with a magic marker or the like. As noted above, the marking band 6 is made of a mesh, so that ink of the magic marker or the like can easily penetrate to the head (see FIG. 7(a) and (b)).

[Step A-8] Six points (referred to as plots) 8 as shown in FIG. 8(a) are marked as markings in the T slice on the monitor, on both sides of the cerebral section. These plots 8 represent the cross sections of the mark sticks 67 and the position of each plot 8 accurately reflects the position of a mark stick 67. Accordingly, from among the combinations of straight lines connecting the left and right plots 8 that run through the center of the hematoma H, the combination is selected, that is most stable and for which there are no obstructions directly near the plots 8, when the device is mounted. Thus, the selected plots 8 are for example plot “a” on the right side and plot “b” on the left side, which are referred to as Ra and Lb, respectively (see FIG. 8(b)).

[Step A-9] The numbers of the left and right mark sticks 67 corresponding to the selected plots 8 as counted from the front are noted down, for example as (R, L)=(3, 4). After this, the positions of the mark sticks 67 corresponding to the selected plots 8 are immediately marked by magic marker or the like on the patient's head. The intersections (Cr, Cl) of the line of the T slice marked in Step A-7 and these mark sticks 67 serve as the mounting location of the device 1 (see FIG. 9(a) and (b)).

[Step A-10] When the marking on the patient is finished, a measurement is performed deliberately on the CT monitor. In this measurement, a line is drawn between Ra and Lb, and the distance to the center of the hematoma (referred to as Hc) (the distance Ra-Hc in the case of a hematoma on the right, and the distance Lb-Hc in the case of a hematoma on the left) is measured and recorded (see FIG. 10(a) and (b)). Each distance can be determined by a computer calculation on the monitor.

When the distances Ra-Lb, Ra-Hc and Lb-Hc have been thus determined, adjustment in accordance with the determined distances is possible without tightening or loosening the fixing frames 3 and 4 when mounting the device 1. Thus, the marking procedure is finished.

It should be noted that it is possible to reuse the marking band 6 after washing it with toluene or the like.

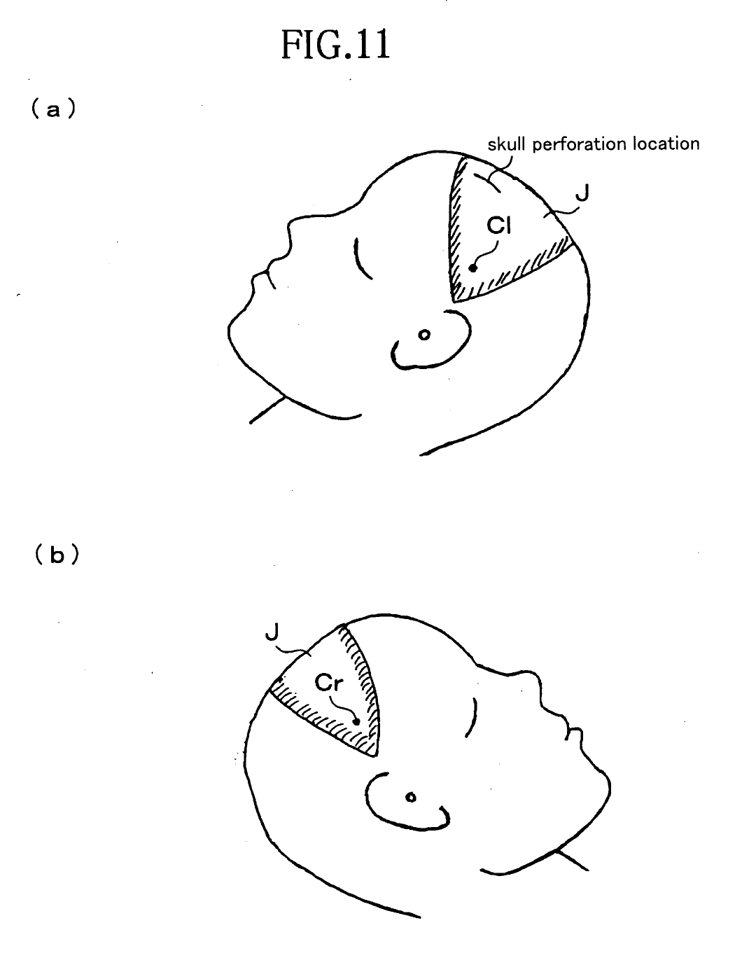

B. The Operation Stage

[Step B-1]: All parts of device 1 and special puncturing needles 71 and 72 are sterilized by gas sterilization or in an autoclave. At this point, the device is not yet assembled.

[Step B-2]: The patient is put in the operating position on the operating table, and after fixing the head, the device mounting locations (Cr, Cl) and the operating field (region within the hatching lines) J, which is the region including a skull perforation location, are sterilized with isodine (see FIG. 11(a) and (b)). Then, the head is covered with cloth after draping, such that the operating field j can be seen.

[Step B-3]: The surgeon makes an incision in the skin at the skull perforation location, drills a hole in the skull, and cuts through the dura mater.

[Step B-4]: Now, the device 1 is assembled under clean conditions. FIG. 2 shows the device 1 in its assembled state.

[Step B-5]: Next, the distance from the point “0” on the scale 22 to the scalp surface matching line 36 (or 46) on the fixing frame 3 (or 4) is set to the distance Ra-Hc (or Lb-Hc). In this situation, the position of the fixing frame 3 (or 4) is set by sliding, and fixed in this position with the fixing screw 34 (or 44). Also, the fixing frame 3 (or 4) for which the distance is set is the frame on the surgery side.

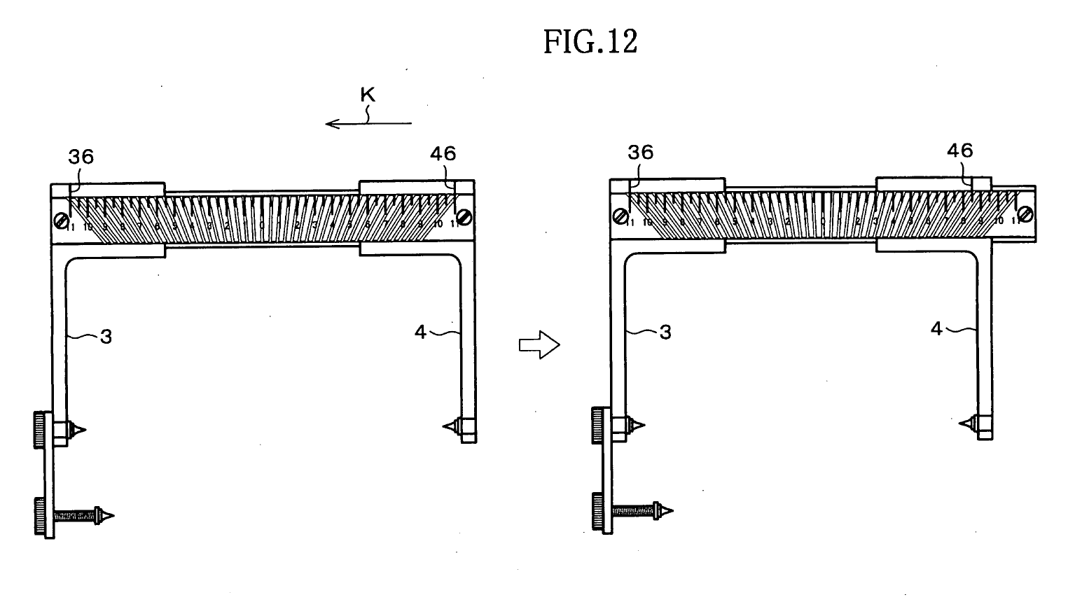

FIG. 12 shows an example of setting the distance from the point “0” on the scale 22 to the scalp surface matching line 46 in accordance with the distance Lb-Hc (8.5 cm in this case) by sliding the left fixing frame 4 in the direction of the arrow K, for the case of a hemorrhage on the left.

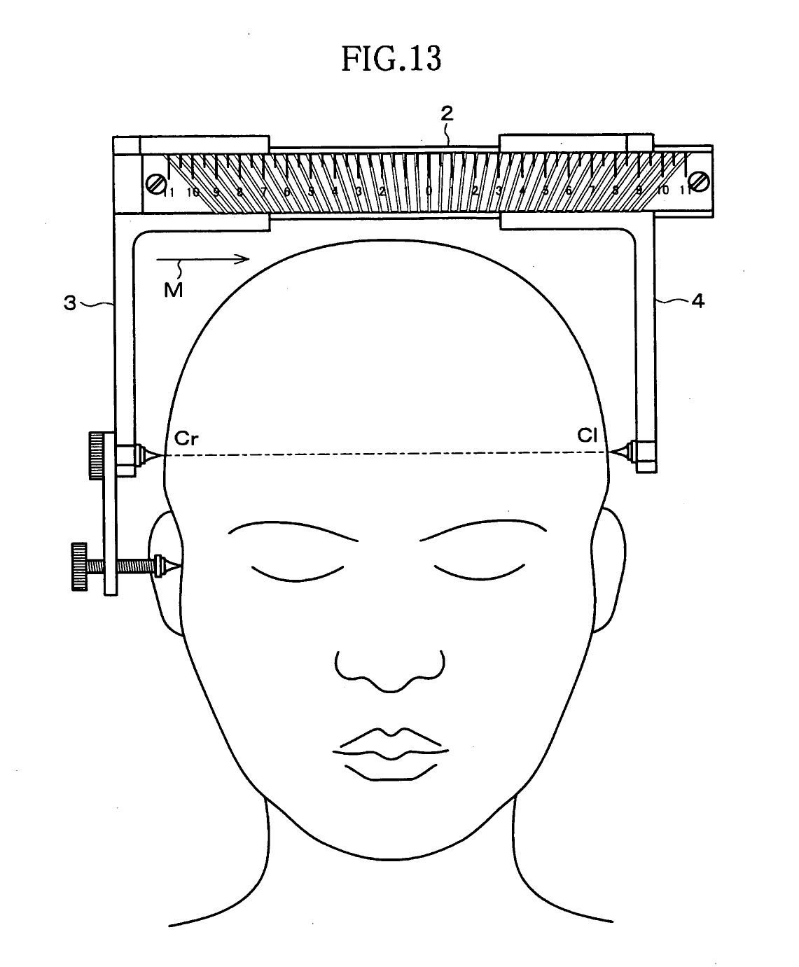

If the left fixing frame 4 is fixed at a certain position as described above, then it stays fixed in this position until the end of the surgery, and the right fixing frame 3 on the opposite side is slid as described below when mounting the device 1 to the patient's head.

[Step B-6]: The fixing needles 31 and 41 are positioned against the point Cr and the point Cl marked on the patient's head, while sliding the right fixing frame 3 on the opposite side in the direction of the arrow M, and after tightening such that the device is sufficiently fixed on the skull, the right fixing frame 3 is fixed at this position with the fixing screw 34 (see FIG. 13).

[Step B-7]: Then, in order to determine which guide groove 21 on the frame 2 with puncture guides to use for the puncturing, the entire device 1 is rotated around the point Cr and the point Cl to the location of the skull perforation, and the position of the optimum guide groove 21 at the location of the skull perforation is ascertained while inserting the puncturing needle 71 into the guide grooves 21 (see FIG. 14). At this time, no puncturing is yet performed.

[Step B-8]: The position of the optimum guide groove 21 at the location of the skull perforation is read from the scale 22 in units of 5 mm from the point “0”, and taken as Sx.

[Step B-9]: The puncturing depth D corresponding to Sx is read from the following puncturing distance table.

| | | |
|---|---|---|
| 0 mm-130 mm | 35 mm-135 mm | 70 mm-148 mm |
| 5 mm-130 mm | 40 mm-136 mm | 75 mm-150 mm |
| 10 mm-130 mm | 45 mm-138 mm | 80 mm-153 mm |
| 15 mm-131 mm | 50 mm-139 mm | 85 mm-155 mm |
| 20 mm-132 mm | 55 mm-141 mm | 90 mm-158 mm |
| 25 mm-132 mm | 60 mm-143 mm | 95 mm-161 mm |
| 30 mm-133 mm | 65 mm-145 mm | 100 mm-164 mm |

In this table, the left side of each field indicates the distance Sx from the point “0” on the scale 22, whereas the right side indicates the puncturing depth D corresponding to that Sx. The puncturing distance D for Sx=0 mm corresponds substantially to the distance F from the frontal upper edge of the frame 2 with puncture guides to the fixing needles 31 and 41.

[Step B-10]: The puncturing depth D is marked with silk threads 73 or the like on two special puncturing needles 71 and 72, one larger than the other (see FIG. 15). Scale divisions of 1 cm are engraved in the puncturing needles 71 and 72, so that this task can be performed easily.

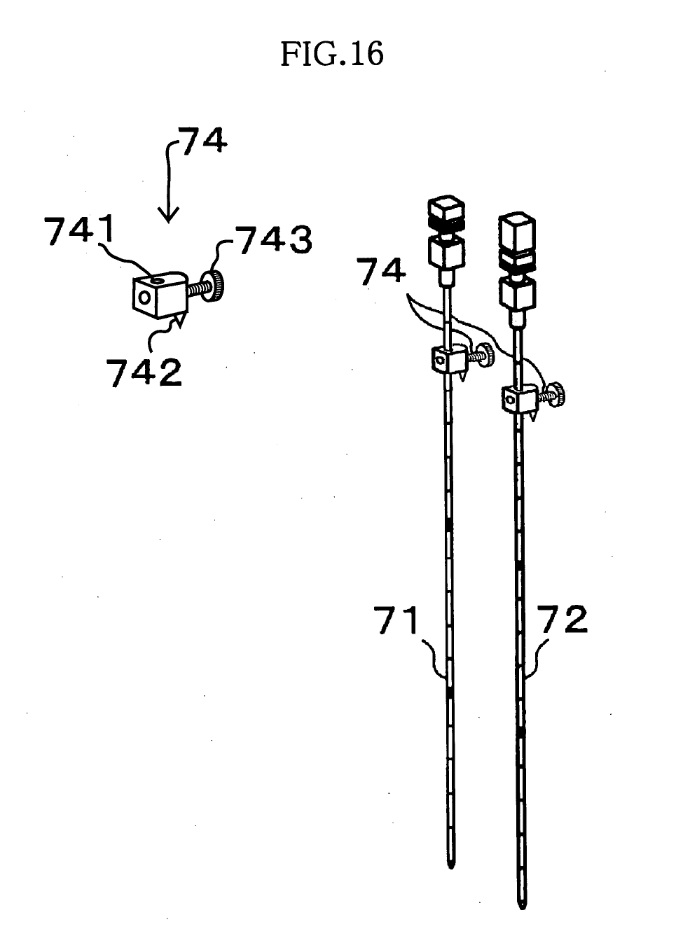

As shown in FIG. 16, it is also possible to mark the position of the puncturing depth D with special stoppers 74 instead of with the silk threads 73. These stoppers 74 include a puncturing needle insertion hole 741, a positioning protrusion 742, and a position fixing knob 743. The puncturing needle 71, 72 is inserted into the puncturing needle insertion hole 741, and the diameter of the puncturing needle hole 741 differs for the puncturing needle 71 and for the puncturing needle 72, in accordance with the diameter of the puncturing needle 71, 72. After the tip of the positioning protrusion 742 has been matched with the position of the puncturing depth D, the position fixing knob 743 is turned, its tip protrudes inside the puncturing needle hole 741, and the stopper 74 is fixed/mounted in this position. Thus, the marking can be easily completed. As shown in FIG. 16, when using puncturing needles 71 and 72 with bold scale divisions every 5 cm, the positioning becomes even easier.

[Step B-11]: The device 1 is rotated to the vicinity of the location of the skull perforation, and the puncturing needle 71 with the smaller diameter is tentatively inserted along the guide groove 21 determined in Step B-8. At this point, no puncturing into the brain is carried out yet. Then, as shown in FIG. 15, the device 1 is firmly fastened (by 3-point fixation) with the auxiliary fixing needle 51 to the head at the optimum position for puncturing.

[Step B-12]: After the puncturing needle 71 with the smaller diameter has been inserted along the guide groove 21 that was determined in Step B-8 to the depth that was marked in advance on the puncturing needle 71 (that is, until the silk thread 73 of the puncturing needle 71 reaches the frontal upper edge of the frame 2 with puncture guides, or until the front tip of the positioning protrusion 742 of the stopper 74 reaches the upper edge of the displacement prevention plate 23), the inner cylinder of the puncturing needle 71 is pulled out.

[Step B-13]: A syringe is attached to the puncturing needle 71, and after it has been confirmed that the tip of the puncturing needle 71 is in the center of the hematoma cavity by evacuation of a small amount of the hematoma, then the outer cylinder of the puncturing needle 71 is pulled out. Thus, a tract to the hematoma cavity is formed.

[Step B-14]: Next, after the puncturing needle 72 with the larger diameter has been actually inserted along the same guide groove 21 up to the depth that has been marked in advance, the inner cylinder of the puncturing needle 72 is pulled out. Then, a syringe of about 10 cc (there is no particular limitation regarding the size, but if it is too large, there is the risk that the hematoma is evacuated too rapidly, leading to more hemorrhaging) is attached to the puncturing needle 72, and the intended amount of hematoma is slowly evacuated.

[Step B-15]: After evacuation of the hematoma is finished, the outer cylinder of the puncturing needle 72 is pulled out. The device 1 is removed from the head, the perforation location is closed, and the surgical procedure is finished.

(4) Modified Embodiment of the Present Invention

In the foregoing, the configuration of the device 1 and the marking band 6 according to the present embodiment as well as a method for using the same were explained, but the above method of use is merely one example, and there is no limitation to the method of use. For example, applications of the device 1 are not limited to evacuation of hematoma or hemorrhages, and the device 1 may also be used for such applications as evacuating cerebrospinal fluid, injecting antibiotics, hematoma dissolution with urokinase, or washing with saline solution.

Also, in the above-described method of use, puncturing needles 71 and 72 were guided to the location of the lesion within the brain, but the device 1 can also be used to guide tools other than the puncturing needles 71 and 72, such as a (silicone) tube that needs to be guided to the location of the lesion within the brain for brain surgery.

Also the configurations of the device and the band of the present invention are not limited to the above-described configurations, various modifications within the scope of the invention are possible, as described below.

[4-A]First Modification Example of the Device

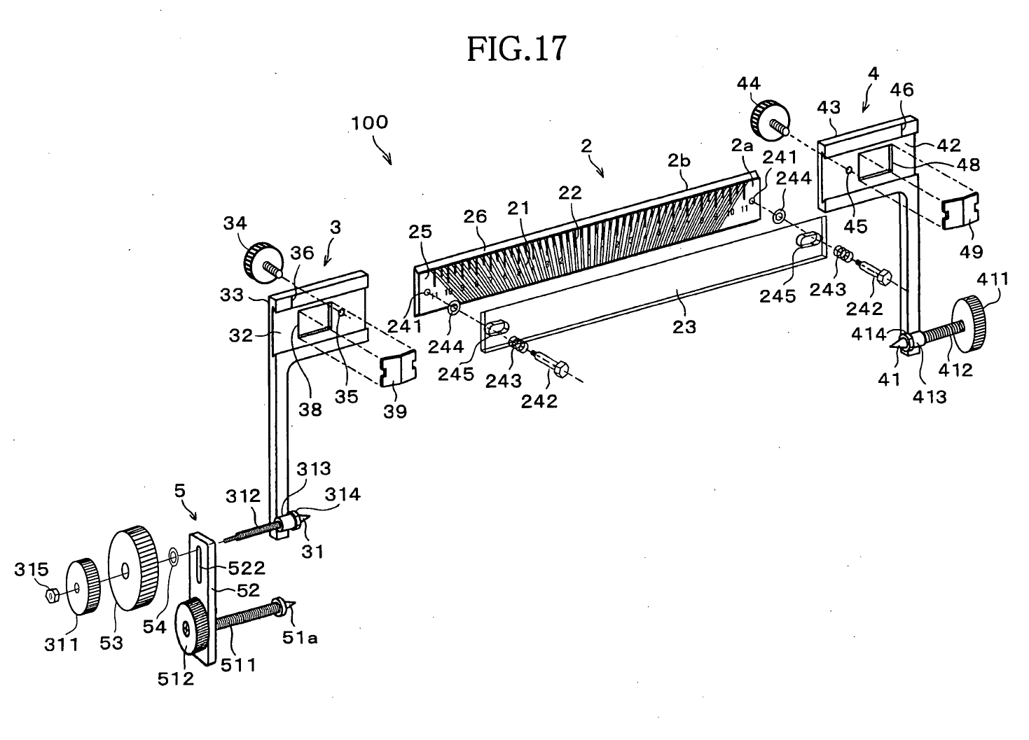

FIG. 17 is an exploded perspective view showing the overall configuration of a simplified stereotactic brain surgery device 100 (referred to below simply as “device 100”) in accordance with another embodiment of the present invention. It should be noted that to simplify explanations, portions that are the same as in the configuration of the above-described device 1 are marked by the same numerals, and their further explanation has been omitted.

In the device 100, the fixing needles 31 and 41 are fastened from both sides by left and right fixing needle fastening knobs 311 and 411. That is to say, male threaded portions are provided on the outer circumference of shafts 312 and 412 of the fixing needles 31 and 41, female threaded portions are provided on the inner circumference of fitting holes 313 and 413 of the fixing frames 3 and 4, the two are screwed together, and fixing needle fastening knobs 311 and 411 are attached to the rear ends of the shafts 312 and 412. Consequently, when the fixing needle fastening knobs 311 and 411 are turned inward, the fixing needles 31 and 41 are fastened on the inner side, and the device 100 can be fastened firmly to the patient's head. A specific fixing method is for example as follows.

First, the fixing needle fastening knobs 311 and 411 are turned, and set to the state in which the fixing needles 31 and 41 are spread furthest apart, that is, to the state in which the brim-shaped plates 314 and 414 of the fixing needles 31 and 41 are closest to the fixing frames 3 and 4. In this state, the fixing needles 31 and 41 are placed against the points Cr and Cl, as in the above-described Step B-6, the fixing frames 3 and 4 are firmly pressed vertically against the patient's skin with both hands, and while the fixing needles 31 and 41 are tightly pressed against the skull, the fixing frame 3 or 4 that is not on the afflicted side (surgery side) is fixed with the fixing screw 34 (or 44). The frame on the afflicted side has already been positioned and fixed, but it may be checked again at this time whether the fixing screw 34 (or 44) has not become loose. The knobs of the fixing screws 34 and 44 are provided with a relatively large diameter as shown in FIG. 17, in order to improve their operability.

Next, in order to strengthen the fixation, fine adjustment is performed with the fixing needle fastening knob 311 (or 411) that is not on the afflicted side. For this, the fixing needle fastening knob on the afflicted side is generally left alone, because there is the risk of deviation from the measured values. That is to say, care is taken that the brim-shaped plate of the fixing needle on the afflicted side does not sink further into the skin. If the brim-shaped plate of the fixing needle on the afflicted side clearly projects outward from the skin surface, then the fixing frame may be pressed on and fixed again, or a fine adjustment may be performed with the fixing needle fastening knob on the afflicted side.

Thus, the fixation of the device 100 on the patient's head can be easily strengthened by fastening and fine-tuning with the fixing needle fastening knobs 311 (or 411). After this, as in the above-described B-11, the device 100 is fixed at three points to the patient's head with the auxiliary fixing needle 51. The fixing of the auxiliary fixing needle 51 to the patient's head is carried out by operating the knob portion 512 and the position fixing knob 53. The position fixing knob 53 should be left somewhat loosened up to this operation, and fastened last. As shown in FIG. 17, in the device 100, a washer 54 is provided between the position fixing knob 53 and the linking arm 52, and the position fixing knob 53 is provided with a relatively large shape in order to improve its operability. The shaft length of the shaft 312 is made longer than that of the male thread 37.

Moreover, the device 100 and the device 1 also differ structurally with regard to the aspects listed below:

(1) Other than the displacement prevention plate 23, the device 100 is made of titanium or a titanium alloy. When the device is mounted during an operation, it is conceivable that tomographic images are taken by MRI, and adverse effects on the images due to mounting the device can be reduced by using a titanium alloy as the device material.

(2) For the material of the displacement prevention plate 23, polycarbonate that has been sterilized in an autoclave is used. Furthermore, the left and right holes 245 of the displacement prevention plate 23 are not true circles but ovals lying on the side. With this structure, deformation (flexure) of the displacement prevention plate 23 can be prevented, even when sterilized in the state that the displacement prevention plate 23 is attached by the screws 242 to the main member 25 of the frame 2 with puncture guides.

(3) The heads of the screws 242 are formed to the same hexagonal shape as the brim-shaped plates 314 and 414 of the fixing needles 31 and 41, and the nuts 315 and 415 for attaching the fixing needle fastening knobs 311 and 411 (the nut 415 is not shown in the figures). This is to ensure that fastening and removing these members can be performed using only a single tool, such as a wrench.

[4-B]Second Modification Example of the Device

It is also possible to apply the following modifications to the device 1, 100.

In the device 1, 100, the guide grooves 21 are engraved as guiding depressions in the frame 2 with puncture guides, but there is no limitation regarding the number, depth, width, length and profile shape of the guide grooves 21, as long as the puncturing needles 71 and 72 can be guided by those grooves. For example, the number of the guide grooves 21 provided on the frame 2 with puncture guides may also be lower than in the configurations of the above-described embodiments. Also, in the above-described embodiment, the diameter of the outer cylinders of the puncturing needles 71 and 72 is 2 mm and 3 mm, respectively, and in this case, it is preferable that the depth of the guide grooves 21 is about 0.6 mm to 1.4 mm, and that the width of the guide grooves 21 is about 1.5 mm to 3 mm. Thus, the preferable depth and width of the guide grooves 21 differ depending on the diameter of the puncture needles 71 and 72 that are used, and there is no particular limitation to them.

Also, there is no particular limitation regarding the length of the guide grooves 21, but in order to guide the puncturing needles 71 and 72 securely and in view of easy handling, it is preferable that the length of the center guide groove 210 is set to about 3 cm to 6 cm.

In the above-described embodiments, the cross-sectional shape of the guide grooves 21 was set to an inverse triangular shape in order to make their fabrication easy, but there is no particular limitation to this. However, when the cross section of the guide grooves 21 is arc-shaped, then the puncturing needles 71 and 72 may enter too deeply into the grooves, and the pressure on the puncturing needles 71 and 72 may become weak, so that it is preferable that the cross section of the guide grooves 21 is polygonal.

Moreover, instead of engraving guide grooves 21 into the frame 2 with puncture guides, it is also possible to provide the guide depressions of the present invention by letting a plurality of walls protrude from the frame 2 with puncture guides. That is to say, it is also possible to let a plurality of walls protrude instead of the guide grooves 21, such that spaces between adjacent walls serve as the guide depressions of the present invention, and the puncturing needles 71 and 72 are inserted into these depressions.

The scales 22 are engraved into the main member 25 of the frame 2 with puncture guides, but the scales 22 may be also displayed on the displacement prevention plate 23 by printed characters or the like. It is also possible to engrave the guide grooves 21 on the displacement prevention plate 23 (on the side facing the main member 25 of the frame 2 with puncture guides).

The displacement prevention plate 23 is pressed against the main member 25 of the frame 2 with puncture guides by the coil spring 243, which serves as a pressuring member, but the pressuring member is not limited to the coil spring 243, and other elastic members such as rubber or plate springs are also possible.

In the above-described device 1, 100, the depressions 32 and 42 in the fixing frames 3 and 4 are fitted to the main member 25 of the frame 2 with puncture guides and slid, but it is also possible to slide the fixing frames 3 and 4 by other configurations (for example by meshing a gear wheel with a rack). Moreover, as long as the fixing frames 3 and 4 can be displaced in the longitudinal direction of the frame 2 with puncture guides and fixed at any desired position on the frame 2 with puncture guides, the fixing frames 3 and 4 do not have to have a configuration in which they are slid.

In the above-described configurations, the auxiliary fixing support 5 is attached to the right fixing frame 3, but it may also be attached to the left fixing frame 4 or provided on both sides.

In the above-described configurations, the fixing needles 31 and 41 are provided as fixing members for fixing the device 1, 100 on the patient's head, but it is also possible to fix the ends of the fixing frames 3 and 4 on the patient's head using an adhesive or sucking disks. In this case, if the ends of the fixing frames 3 and 4 and the portions fixed to the patient's head by an adhesive or the like are linked by a shaft, then the entire device 1, 100 can be rotated around this shaft to the location of the skull perforation.

There is also no particular limitation to the material of the device 1, 100, and it is possible to use materials such as plastics or metals other than stainless steel or titanium (alloys), but a material suitable for sterilization is preferable. Also the displacement prevention plate 23 may be made of other materials than acrylic resin or polycarbonate.

Furthermore, design changes of the shape, size, length etc. of each member to achieve a suitable shape, size, length etc. are possible. For example, it is possible to change the design of the tip shape and size of the fixing needles 31 and 41 and the auxiliary fixing needle 51 to a suitable shape and size, or to change the shaft length of the screw portion 511 of the auxiliary fixing needle 51 to a suitable length. Furthermore, the tip 51a of the auxiliary fixing needle 51 may be configured such that it does not rotate in cooperation with a rotation of the shaft of the screw portion 511. With this configuration, the tip 51a of the auxiliary fixing needle 51 is prevented from becoming entangled in the covering cloth or the like, when the device 1, 100 is fixed at three points with the auxiliary fixing needle 51.

[4-C]Modification Example of the Band

The marking band 6 has a total of twelve mark sticks 67, six on each of the two bands 61 and 62, but the number of mark sticks 67 is not limited to this. It should be noted that when the number of mark sticks 67 is too low, then there is the risk that it is not possible to select a suitable plot 8 when selecting the left and right plots 8 in Step A-8, and it may be necessary to retake the CT. On the other hand, when the number of mark sticks 67 is too high, then the possibility increases that an error occurs when marking the positions of the mark sticks 67 corresponding to the plots 8 selected in Step A-9, so that the number of mark sticks 67 is preferably about 3 to 8 each on the left and right and more preferably about 4 to 7.

The mark sticks 67 serving as marker members are made of an aluminum alloy whose aluminum content is 99.77%, and further contains titanium, iron and silicon as components, but there is no particular limitation to the aluminum content when using an aluminum alloy. In order to provide the mark sticks 67 with sufficient flexibility to arrange them along the head's surface, it is preferable that the aluminum content is high (for example at least 70%).

Other than aluminum alloy, it is possible to use any metal or alloy that has a low X-ray permeability and produces few artifacts when taking CTs, such as aluminum, stainless steel, brass, copper or nichromium, for the metal rods (or metal wires) constituting the mark sticks 67. Moreover, it is also possible to use materials other than metal, provided that those materials have low X-ray permeability and produce few artifacts when taking CTs, such as processed animal bone or teeth (ivory, human bone, fishbone or the like).

Moreover, the mark sticks 67 may also include other materials than the above. For example, the metal rods (or metal wires) made of any of the above-noted metals may be covered with an insulator.

The length of the mark sticks 67 is about 5 cm, and the distance between the mark sticks 67 is about 2 cm, but there is no limitation to this. The mark sticks 67 are formed into thin rods, but when they are too thin, it becomes difficult to insert them into the main member 65 of the band. On the other hand, when the metal roods are too thick, artifacts tend to occur, so that it is preferable that the cross-sectional diameter of the mark sticks 67 is about 1 mm to 2 mm. That is to say, it is preferable that the material and the thickness of the mark sticks 67 are such that the mark sticks 67 have enough flexibility to bring them into substantially full contact with the surface of the head by simply pressing them against the patient's head, and it is preferable that the mark sticks 67 are not too soft and that their material and thickness is such that their X-ray permeability is low and that artifacts do not tend to occur when taking CTs.

The two bands 61 and 62 constituting the marking bands 6 are made by connecting the two bands 61 and 62 together with the cloth tapes 63 and 64, but there is no limitation to this, and it is also possible to make the marking band 6 by connecting the two bands 61 and 62 together by other means, such as paper tapes.

There is no particular limitation regarding the material of the two bands 61 and 62, but a material with low stretchability in longitudinal direction is preferable, because there is the risk that the positions of the mark sticks 67 shift and accurate marking becomes impossible when the material is stretchable in the longitudinal direction. Moreover, a material that has enough flexibility to enable fitting on and wrapping around the patient's head is preferable.

(5) Advantages of the Embodiments

Regarding the advantages of the device 1 according to the present invention over the related art, the following explanations separately discuss the advantages over Komai-type devices and the advantages over the device disclosed in JP H11-137568A.

[5-A] Advantages Over Komai-Type Devices

(1) With a Komai-type device, it is necessary to mount the device already during the stage of taking the CT, whereas with the device 1, there is no necessity to mount the device 1 when taking the CT, and it is enough to mount the device 1 in the operation room after taking the CT. Therefore, the device 1 is only used under sterilized conditions, which makes it more hygienic and reduces the possibility of infections greatly.

(2) Moreover, the device 1 is mounted in the operating room under anesthesia, so that it is less stressful on the patient.

(3) Since its configuration is simple, the device 1 can be assembled easily, so that the operating time can be shortened accordingly, and surgical invasion becomes lower. Moreover, when there are complications or accidents during the operation, such as respiratory insufficiency, then countermeasures can be taken more quickly, because the device 1 can be easily removed.

[5-B]Advantages Over the Device Disclosed in JP H11-137568A

(1) The tips 71a and 72a of the puncturing needles 71 and 72 are guided by the guided grooves 21 on the frame 2 with puncture guides to the location of the lesion within the brain, so that other members such as an arc-shaped frame are not necessary during the puncturing procedure. Thus, the procedure is easy in this respect, and has excellent operability. Furthermore, a curved member, such as an arc-shaped frame, is not necessary, so that sterilization is easy and the device is hygienic, and the device can be fabricated more easily and at lower cost.

(2) Even while one of the puncturing needles 71 and 72 with different diameters is inserted into the brain, that puncturing needle 71 or 72 is sandwiched by the main member of the frame 2 for puncture guides and the displacement prevention plate 23 and displacement can be prevented reliably. Consequently, destruction of tissue is minimal, so that a device configuration with high security preventing further bleeding is obtained. Moreover, a plurality of puncturing needles 71 and 72 of different diameters can be guided by this configuration, so that it is possible to first provide a tract (path) to the hematoma cavity with the thinner puncturing needle 71, and then insert into that tract the puncturing needle 72 for evacuating the hematoma with the same device 1. In this surgical procedure, further bleeding tends not to occur and it is gentle on the surrounding brain tissue, so that the device configuration also provides high security regarding this aspect.

(3) The auxiliary fixing support 5 has a simple configuration and it is easy to adjust the angle θ and the distance E, so that the position where the device is fixed to the patient's head can be freely selected within an adjustment range with the auxiliary fixing needle 51. That is to say, the range over which fixing with the auxiliary fixing needle 51 is possible is large, and accordingly, it is possible to select a safer fixing position.

(4) The principal members are of substantially linear shape, so that fabrication of the device is accordingly inexpensive, and sterilization can be performed reliably, making it hygienic.

The invention may be embodied in other forms without departing from the spirit or essential characteristics thereof. The embodiments disclosed in this application are to be considered in all respects as illustrative and not limiting. The scope of the invention is indicated by the appended claims rather than by the foregoing description, and all changes which come within the meaning and range of equivalency of the claims are intended to be embraced therein.

### INDUSTRIAL APPLICABILITY

The present invention relates to simplified stereotactic brain surgery devices used for surgery on cerebral diseases such as hypertensive cerebral hemorrhages, brain tumors, brain abscesses, hydrocephalus, or cerebral cysts, and to bands used for determining the position at which such a device is mounted on a patient's head, and can be used for various kinds of brain surgery, such as evacuation surgery of hematoma or hemorrhages, evacuation of cerebrospinal fluid, injecting antibiotics, hematoma dissolution with urokinase, or washing with saline solution.

1. A simplified stereotactic brain surgery device, comprising:
a guide frame for guiding a puncturing needle's tip to a predetermined location within a brain; and
left and right fixing frames, each having a fixing member for fixing the device to the patient's head, the fixing frames being displaceable in a longitudinal direction of the guide frame;
wherein the guide frame is provided with a plurality of guide depressions that guide the puncturing needle's tip to a point on the line connecting the left and right fixing members.

2. The simplified stereotactic brain surgery device according to claim 1, wherein the guide frame is provided with a scale.

3. The simplified stereotactic brain surgery device according to claim 1, wherein the guide frame comprises a displacement prevention member for preventing displacement of the puncturing needle when the puncturing needle is guided by the guide depressions.

4. The simplified stereotactic brain surgery device according to claim 3, further comprising a pressing member that presses the displacement prevention member against a main member of the guide frame.

5. The simplified stereotactic brain surgery device according to any of claims 1, wherein an auxiliary fixing member for aiding fixation of the device on the patient's head is provided on at least one of the left and right fixing frames.

6. The simplified stereotactic brain surgery device according to claim 5, wherein the auxiliary fixing member comprises an auxiliary fixing portion for aiding fixation of the device on the patient's head, a linking member linking the auxiliary fixing portion and the fixing frame at an adjustable angle with the fixing member at the center, and a distance adjustment member for adjusting a distance between the auxiliary fixing portion and that fixing member.

7. A band that is used prior to brain surgery using the simplified stereotactic brain surgery device according to any of claims 1, to determine a position where the device is mounted to the patient's head and to mark this position, the band having a plurality of marker members that are arranged at opposing positions when the band is wrapped around the patient's head.

8. The band according to claim 7, wherein the marker members comprise aluminum or an alloy thereof, stainless steel, brass, copper, nichromium, processed animal bone, or tooth.

9. The band according to claim 7, whose main member is mesh-shaped.
